SEQUENCE LISTING (46 sequences)

SEQ ID NO: 1 - RNA, 102 nt, synthetic construct
  misc_feature 1..102: note = Description of Artificial Sequence: Synthetic polynucleotide
  N_region 1..20
  source 1..102: mol_type = other RNA
nnnnnnnnnnnnnnnnnnnngttttagagctagaaatagcaagttaaaataaggctagtccgttatcaacttgaaaaagtggcaccgagtcggtgctttttt

SEQ ID NO: 2 - RNA, 82 nt, synthetic construct
  misc_feature 1..82: note = Description of Artificial Sequence: Synthetic oligonucleotide
  source 1..82: mol_type = other RNA
gttttagagctagaaatagcaagttaaaataaggctagtccgttatcaacttgaaaaagtggcaccgagtcggtgctttttt

SEQ ID NO: 3 - DNA, 3974 bp, Streptococcus pyogenes
  source 1..3974: mol_type = genomic DNA
atggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaa

SEQ ID NO: 4 - protein, 1423 aa, Streptococcus pyogenes
MDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 5 - RNA, 105 nt, synthetic construct
  misc_feature 1..105: note = Description of Artificial Sequence: Synthetic polynucleotide
  N_region 2..22
  source 1..105: mol_type = other RNA
gnnnnnnnnnnnnnnnnnnnnngttttagtactctggaaacagaatctactaaaacaaggcaaatgccgtgtttatctcgtcaacttgttggcgaagattttttt

SEQ ID NO: 6 - RNA, 83 nt, synthetic construct
  misc_feature 1..83: note = Description of Artificial Sequence: Synthetic oligonucleotide
  source 1..83: mol_type = other RNA
gttttagtactctggaaacagaatctactaaaacaaggcaaatgccgtgtttatctcgtcaacttgttggcgaagattttttt

SEQ ID NO: 7 - DNA, 3345 bp, Staphylococcus aureus
  source 1..3345: mol_type = genomic DNA
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaa

SEQ ID NO: 8 - protein, 1114 aa, Staphylococcus aureus
MAPKKKRKVGIHGVPAAKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKGSYPYDVPDYAYPYDVPDYAYPYDVPDYA

SEQ ID NO: 9 - DNA, 20 bp, synthetic construct
  misc_feature 1..20: note = Synthetic oligonucleotide
  source 1..20: mol_type = other DNA
cacttcagctagaaaccagt

SEQ ID NO: 10 - DNA, 20 bp, synthetic construct
  misc_feature 1..20: note = Synthetic oligonucleotide
  source 1..20: mol_type = other DNA
gtagtctcagtgttcagaca

SEQ ID NO: 11 - DNA, 20 bp, synthetic construct
  misc_feature 1..20: note = Synthetic oligonucleotide
  source 1..20: mol_type = other DNA
taggaaaagatgtaactagg

SEQ ID NO: 12 - DNA, 20 bp, synthetic construct
  misc_feature 1..20: note = Synthetic oligonucleotide
  source 1..20: mol_type = other DNA
gtcgtaggatcagcacaaag

SEQ ID NO: 13 - DNA, 20 bp, synthetic construct
  misc_feature 1..20: note = Synthetic oligonucleotide
  source 1..20: mol_type = other DNA
gaaaaacactagtttcacca

SEQ ID NO: 14 - DNA, 20 bp, synthetic construct
  misc_feature 1..20: note = Synthetic oligonucleotide
  source 1..20: mol_type = other DNA
ttggccatctaatgaacctg

SEQ ID NO: 15 - DNA, 58 bp, synthetic construct
  misc_feature 1..58: note = Synthetic oligonucleotide
  source 1..58: mol_type = other DNA
aaaacccaaacacttcagctagaaaccagtaggaatctaaaggacagtaataattttt

SEQ ID NO: 16 - DNA, 66 bp, synthetic construct
  misc_feature 1..66: note = Synthetic oligonucleotide
  source 1..66: mol_type = other DNA
ctagaaaaattattactgtcctttagattcctactggtttctagctgaagtgtttgggttttgtac

SEQ ID NO: 17 - DNA, 57 bp, synthetic construct
  misc_feature 1..57: note = Synthetic oligonucleotide
  source 1..57: mol_type = other DNA
aactaaaccacccctaaaacttggccatgtctgaacactgagactactaatactttg

SEQ ID NO: 18 - DNA, 65 bp, synthetic construct
  misc_feature 1..65: note = Synthetic oligonucleotide
  source 1..65: mol_type = other DNA
ctagcaaagtattagtagtctcagtgttcagacatggccaagttttaggggtggtttagttgtac

SEQ ID NO: 19 - DNA, 57 bp, synthetic construct
  misc_feature 1..57: note = Synthetic oligonucleotide
  source 1..57: mol_type = other DNA
tatacgagatggaaatggacattgccacgtttatggccaaggttttcaatataaaac

SEQ ID NO: 20 - DNA, 65 bp, synthetic construct
  misc_feature 1..65: note = Synthetic oligonucleotide
  source 1..65: mol_type = other DNA
ctaggttttatattgaaaaccttggccataaacgtggcaatgtccatttccatctcgtatagtac

SEQ ID NO: 21 - DNA, 57 bp, synthetic construct
  misc_feature 1..57: note = Synthetic oligonucleotide
  source 1..57: mol_type = other DNA
gtagtactgcttggccatctaatgaacctgaggaaaaagaaagaacagagtgataat

SEQ ID NO: 22 - DNA, 65 bp, synthetic construct
  misc_feature 1..65: note = Synthetic oligonucleotide
  source 1..65: mol_type = other DNA
ctagattatcactctgttctttctttttcctcaggttcattagatggccaagcagtactacgtac

SEQ ID NO: 23 - DNA, 127 bp, synthetic construct
  misc_feature 1..127: note = Synthetic oligonucleotide
  source 1..127: mol_type = other DNA
tcatgtggtcggggtagcggctgaagcactgcacgccgtacgtcagggtggtcacgagggtgggccagggcacgggcagcttgccggtggtgcagatgaacttcagggtcagcttgccgtaggtggc

SEQ ID NO: 24 - DNA, 20 bp, synthetic construct
  misc_feature 1..20: note = Synthetic oligonucleotide
  source 1..20: mol_type = other DNA
ttctccaaggattgggactg

SEQ ID NO: 25 - DNA, 18 bp, synthetic construct
  misc_feature 1..18: note = Synthetic oligonucleotide
  source 1..18: mol_type = other DNA
gctgatcctacgactacg

SEQ ID NO: 26 - DNA, 25 bp, synthetic construct
  misc_feature 1..25: note = Synthetic oligonucleotide
  source 1..25: mol_type = other DNA
ggctgaatccttggtaaatatgaag

SEQ ID NO: 27 - DNA, 20 bp, synthetic construct
  misc_feature 1..20: note = Synthetic oligonucleotide
  source 1..20: mol_type = other DNA
gcagcatgaaagagccccac

SEQ ID NO: 28 - DNA, 19 bp, synthetic construct
  misc_feature 1..19: note = Synthetic oligonucleotide
  source 1..19: mol_type = other DNA
gctggagagagagggagtg

SEQ ID NO: 29 - DNA, 20 bp, synthetic construct
  misc_feature 1..20: note = Synthetic oligonucleotide
  source 1..20: mol_type = other DNA
cactgctcacaggaggtgaa

SEQ ID NO: 30 - DNA, 19 bp, synthetic construct
  misc_feature 1..19: note = Synthetic oligonucleotide
  source 1..19: mol_type = other DNA
gagccagtaaaatgtccac

SEQ ID NO: 31 - DNA, 32 bp, synthetic construct
  misc_feature 1..32: note = Synthetic oligonucleotide
  source 1..32: mol_type = other DNA
atatataagcttcccaacccaacaacaagtct

SEQ ID NO: 32 - DNA, 36 bp, synthetic construct
  misc_feature 1..36: note = Synthetic oligonucleotide
  source 1..36: mol_type = other DNA
atatatgaattccacagctgttgttagtttccaccg

SEQ ID NO: 33 - DNA, 28 bp, synthetic construct
  misc_feature 1..28: note = Synthetic oligonucleotide
  source 1..28: mol_type = other DNA
atatatgaattcgaaagatctttgagga

SEQ ID NO: 34 - DNA, 37 bp, synthetic construct
  misc_feature 1..37: note = Synthetic oligonucleotide
  source 1..37: mol_type = other DNA
atatatctcgagcagcccagaacatttaacttaacac

SEQ ID NO: 35 - DNA, 19 bp, synthetic construct
  misc_feature 1..19: note = Synthetic oligonucleotide
  source 1..19: mol_type = other DNA
ctagaaggcacagtcgagg

SEQ ID NO: 36 - DNA, 20 bp, synthetic construct
  misc_feature 1..20: note = Synthetic oligonucleotide
  source 1..20: mol_type = other DNA
tagggacggacaaagagctg

SEQ ID NO: 37 - DNA, 20 bp, synthetic construct
  misc_feature 1..20: note = Synthetic oligonucleotide
  source 1..20: mol_type = other DNA
cactgctcacaggaggtgaa

SEQ ID NO: 38 - DNA, 19 bp, synthetic construct
  misc_feature 1..19: note = Synthetic oligonucleotide
  source 1..19: mol_type = other DNA
gagccagtaaaatgtccac

SEQ ID NO: 39 - DNA, 20 bp, synthetic construct
  misc_feature 1..20: note = Synthetic oligonucleotide
  source 1..20: mol_type = other DNA
tagggacggacaaagagctg

SEQ ID NO: 40 - DNA, 21 bp, synthetic construct
  misc_feature 1..21: note = Synthetic oligonucleotide
  source 1..21: mol_type = other DNA
ccaaggattcagccaattaaa

SEQ ID NO: 41 - DNA, 20 bp, synthetic construct
  misc_feature 1..20: note = Synthetic oligonucleotide
  source 1..20: mol_type = other DNA
gcctccaacctgaatcttga

SEQ ID NO: 42 - DNA, 25 bp, synthetic construct
  misc_feature 1..25: note = Synthetic oligonucleotide
  source 1..25: mol_type = other DNA
aaaagagaaacaacattacagatcc

SEQ ID NO: 43 - DNA, 25 bp, synthetic construct
  misc_feature 1..25: note = Synthetic oligonucleotide
  source 1..25: mol_type = other DNA
tcacccacactgtgcccatctacga

SEQ ID NO: 44 - DNA, 25 bp, synthetic construct
  misc_feature 1..25: note = Synthetic oligonucleotide
  source 1..25: mol_type = other DNA
cagcggaaccgctcattgccaatgg

SEQ ID NO: 45 - DNA, 24 bp, synthetic construct
  misc_feature 1..24: note = Synthetic oligonucleotide
  source 1..24: mol_type = other DNA
ccagtattgatcgggagagccgga

SEQ ID NO: 46 - DNA, 24 bp, synthetic construct
  misc_feature 1..24: note = Synthetic oligonucleotide
  source 1..24: mol_type = other DNA
cttttcctccagagcccgactcgc

(19) United States
(12) Patent Application Publication
(10) Pub. No.: US 2023/0407279 A1
(43) Pub. Date: Dec. 21, 2023

(54) CRISPR/CAS9 TARGETED EXCISION OF THE INTRONIC CTG18.1 TRINUCLEOTIDE REPEAT EXPANSION OF TCF4 AS A THERAPY IN FUCHS' ENDOTHELIAL CORNEAL DYSTROPHY

(71) Applicant: Avellino Lab USA, Inc., Menlo Park, CA (US)

(72) Inventor: Tara MOORE, Ballyclare (GB)

(21) Appl. No.: 18/192,590

(22) Filed: Mar. 29, 2023

Related U.S. Application Data
(63) Continuation-in-part of application No. PCT/US21/52592, filed on Sep. 29, 2021.
(60) Provisional application No. 63/084,774, filed on Sep. 29, 2020.

(51) Int. Cl. C12N 9/22; A61P 27/02

(52) U.S. Cl. CPC C12N 9/22; A61P 27/02; C12N 2310/20

(57) ABSTRACT

The present disclosure relates to Clustered Regularly Interspaced Short Palindromic Repeats (CRISPR)/CRISPR associated protein 9 (Cas9) systems, and methods of use thereof for gene editing.

### RELATED APPLICATIONS

This application is a continuation-in-part application of the international patent application No. PCT/US2021/052592, filed Sep. 29, 2021, which claims priority to U.S. Provisional Patent Application No. 63/084,774, filed Sep. 29, 2020, both of which are incorporated by reference herein in their entireties.

### REFERENCE TO AN ELECTRONIC SEQUENCE LISTING

The electronic sequence listing, entitled “SequenceListing.xml” created on or about Mar. 29, 2023 with a file size of about 69 KB, contains the sequence listing for this application and is hereby incorporated by reference in its entirety.

### FIELD OF THE INVENTION

The present disclosure relates to Clustered Regularly Interspaced Short Palindromic Repeats (CRISPR)/CRISPR associated protein 9 (Cas9) systems, and methods of use thereof for gene editing or preventing, ameliorating or treating a disease associated with a repeat expansion in a subject.

### BACKGROUND OF THE INVENTION

Fuchs' endothelial corneal dystrophy (FECD; [MIM: 613267]) is a progressive age-related degeneration of the corneal endothelium affecting 5% of the population over 40 years of age in the United States (Lorenzetti et al., Central cornea guttata. Incidence in the general population. Am J Ophthalmol. 1967; 64:1155-1158) and represents the leading indication for corneal transplantation in the United States (US) and other western countries (Al-Yousuf et al., Penetrating keratoplasty: indications over a 10 year period. Br J Ophthalmol. 2004; 88(8):998-1001; Ghosheh et al., Trends in penetrating keratoplasty in the United States 1980-2005. Int Ophthalmol. 2008; 28(3):147-153; Kang et al., Trends in the indications for penetrating keratoplasty, 1980-2001. Cornea. 2005; 24(7):801-3; Matthaei et al., Changing Indications in Penetrating Keratoplasty: A Systematic Review of 34 Years of Global Reporting. Transplantation. 2016). The early stages of FECD are asymptomatic but progressive endothelial cell loss by premature senescence and apoptosis (Kenney et al., Characterization of the Descemet's membrane/posterior collagenous layer isolated from Fuchs' endothelial dystrophy corneas. Exp Eye Res. 1984; 39(3):267-77), and Descemet's membrane thickening, result in extensive guttae, endothelial cell loss and eventually corneal edema and subsequent loss of vision. The corneal endothelium is a non-regenerative monolayer of hexagonal cells on the inner surface of the cornea which functions as a highly metabolically active pump to maintain corneal stromal dehydration and clarity for clear vision. During adulthood, the corneal endothelial cell density slowly decreases (Bourne et al. (1997) Central corneal endothelial cell changes over a ten-year period. Invest Ophthalmol Vis Sci.; 38(3):779-82; Murphy et al. (1984) Prenatal and postnatal cellularity of the human corneal endothelium. A quantitative histologic study. Invest Ophthalmol Vis Sci.; 25(3):312-22; Yong et al. (2013) Long-term Evaluation of Endothelial Cell Changes in Fuchs Corneal Dystrophy: The Influence of Phacoemulsification and Penetrating Keratoplasty. Korean J Ophthalmol.; 27(6): 409-415) and the loss is compensated by active sliding and enlargement of adjacent cells (Joyce N C. (2005) Cell cycle status in human corneal endothelium. Exp Eye Res.; 81(6):629-638). Due to aging or endothelial trauma/disease, when the density falls below the threshold, endothelial decompensation occurs as the passive leaking of corneal stroma can no longer be compensated by cell enlargement alone. This results in irreversible corneal edema and haze in the visual axis (B. Dooren. (2006) The corneal endothelium reflected: Studies on surgical damage to the corneal endothelium and on endothelial specular microscopy. Erasmus University Medical Center, Rotterdam, Netherlands). The clinical hallmark of FECD which becomes clinically evident in the fourth and fifth decades of life is the development of focal excrescences and guttae in Descemet's membrane (Klintworth G. Corneal dystrophies. Orphanet J Rare Dis. 2009; 4(1):7). These guttae are easily visualised by slit-lamp biomicroscopy. The process is often accelerated by cataract surgery which can result in irreversible corneal oedema in FECD (website: www.aop.org.uk/ot/CET/2015/10/19/managing-cataract-patients-with-fuchs-endothelial-dystrophy/article).

Currently, the only definitive treatment option to restore vision in FECD is corneal allogenic transplantation; either full-thickness penetrating keratoplasty or lamellar transplant involving DASEK or DMEK (Price et al., Descemet's Stripping Automated Endothelial Keratoplasty Outcomes Compared with Penetrating Keratoplasty from the Cornea Donor Study. Ophthalmology. 2010; 117(3):438-44; Dapena et al., Endothelial keratoplasty: DSEK/DSAEK or DMEK—the thinner the better? Curr Opin Ophthalmol. 2009; 20(4):299-307; Tourtas et al., Descemet membrane endothelial keratoplasty versus descemet stripping automated endothelial keratoplasty. Am J Ophthalmol. 2012; 153 (6):1082-90). Despite advances in surgical techniques corneal transplantation is still associated with significant complications: rejection, graft failure, glaucoma and graft dehiscence. Despite efforts to increase corneal donation, there is a global shortage of tissue for transplantation with only 1 cornea available for every 70 needed which is compounded by increasing demand given the ageing global population (Gain et al., Global Survey of Corneal Transplantation and Eye Banking. JAMA Ophthalmol. 2016 February; 134(2):167-73). There is a pressing need to develop alternative treatments, such as corneal bioengineering, gene and cell-based therapies.

FECD, in most cases, is inherited as an autosomal dominant trait but there are genetic and environmental modifiers that determine the degree to which members of the same family express the disease (Magovern et al. (1979) Inheritance of Fuchs' combined dystrophy. Ophthalmology 86: 1897-1923; Krachmer et al. (1980) Inheritance of endothelial dystrophy of the cornea. Ophthalmologica 181: 301-313; Rosenblum et al. (1980) Hereditary Fuchs' Dystrophy. Am J Ophthalmol 90: 455-462). An early onset form of FECD has been linked to COL8A2 (Collagen type VIII) mutations and is the rarer form of disease (Gottsch et al., Inheritance of a novel COL8A2 mutation defines a distinct early-onset subtype of fuchs corneal dystrophy. Investigative ophthalmology & visual science. 2005; 46(6):1934-9). The late onset form is more common and is genetically heterogeneous with mutations in SLC4A11 (Sodium bicarbonate transporter like protein 11) (Vithana et al., SLC4A11 mutations in Fuchs endothelial corneal dystrophy. Hum Mol Genet. 2008; 17(5):656-66), TCF8 (Zinc finger E-box binding homeobox 1) (23. Riazuddin S A, Zaghloul N A, Al-Saif A, Davey L, Diplas B H, et al. (2010) Missense mutations in TCF8 cause late-onset fuchs corneal dystrophy and interact with FECD4 on chromosome 9p. Am J Hum Genet 86: 45-53), LOXHD1 (Lipoxygenase Homology Domains 1) (Riazuddin et al., Mutations in LOXHD1, a recessive-deafness locus, cause dominant late-onset Fuchs corneal dystrophy. Am J Hum Genet. 2012; 90(3):533-9), and AGBL1 (ATP/GTP Binding Protein Like 1)(Riazuddin S A, Vasanth S, Katsanis N, Gottsch J D. Mutations in AGBL1 cause dominant late-onset Fuchs corneal dystrophy and alter protein-protein interaction with TCF4. Am J Hum Genet. 2013; 93 (4):758-64) genes linked to a small proportion of cases. Recent linkage and genome-wide association (GWA) studies have linked FECD with an expanded trinucleotide repeat (CTG18.1 locus) in the second intron of transcription factor 4 (TCF4, MIM 602272) which is strongly associated with late onset FECD and accounts for 70% of the FECD cases in the USA (Wieben et al., A common trinucleotide repeat expansion within the transcription factor 4 (TCF4, E2-2) gene predicts Fuchs corneal dystrophy. PLoS One. 2012; 7(11):e49083; Wieben et al., Comprehensive assessment of genetic variants within TCF4 in Fuchs' endothelial corneal dystrophy. Invest Ophthalmol Vis Sci. 2014; 55(9):6101-7; Baratz et al., E2-2 protein and Fuchs's corneal dystrophy. N Engl J Med. 2010; 363(11):1016-24; Li et al., Replication of TCF4 through association and linkage studies in late-onset Fuchs endothelial corneal dystrophy. PLoS One. 2011, 6(4):e18044; Mootha wt al. (2014) Association and familial segregation of CTG18.1 trinucleotide repeat expansion of TCF4 gene in Fuchs' endothelial corneal dystrophy. Investigative Ophthalmology & Visual Science, 55, 33-42; Xing et al. (2014) Transethnic replication of association of CTG18.1 repeat expansion of TCF4 gene with Fuchs' corneal dystrophy in Chinese implies common causal variant. Investigative Ophthalmology & Visual Science, 55, 7073-7078; Soliman et al. (2015) Correlation of severity of Fuchs endothelial corneal dystrophy with triplet repeat expansion in TCF4. JAMA Ophthalmology, 133, 1386-1391). The recent GWA study identified a strong association between SNPs in TCF4 and FECD with the most highly associated SNP (rs613872) in linkage disequilibrium with CTG18.1; a (CTG·CAG)ₙ trinucleotide repeat (TNR) of TCF4. One copy of the risk allele in rs613872 results in an odds ratio (OR) of 5.5 whereas those with two copies had an OR of 30. The association between the CTG18.1 TNR and FECD has been confirmed in large numbers of ethnically diverse patients worldwide and a monoallelic or biallelic expansion of CTG18.1 of (CTG·CAG)_{n>40} is significantly correlated with FECD (Vasanth et al., Expansion of CTG18.1 Trinucleotide Repeat in TCF4 Is a Potent Driver of Fuchs' Corneal Dystrophy. Invest Ophthalmol Vis Sci. 2015 July; 56(8):4531-6). Microsatellites or expansion of nucleotide repeats occur naturally in the human genome and have important roles in genome evolution and function (Rohilla and Gagnon, RNA biology of disease-associated microsatellite repeat expansions. Acta Neuropathol Commun. 2017 Aug. 29; 5(1):63). However, the expansion of nucleotide repeats is associated with over 20 neurological diseases including myotonic dystrophy, Friedreich's ataxia and Huntington's disease (Rohilla and Gagnon, 2017). Microsatellite expansions cause disease through two broad molecular mechanisms: loss-of-function for the associated gene or gain-of-function for the repeat expansion sequence (Rohilla and Gagnon, 2017). There are several mechanisms by which the intronic trinucleotide repeat expansion in TCF4 leads to the development of FECD—a direct effect on TCF4 expression, production of toxic repeat-associated non-ATG (RAN) translation products and changes in RNA splicing. RNA toxicity is a common pathogenic mechanism in microsatellite disorders and expanded CUG-repeat RNA transcripts accumulate in nuclear foci in FECD corneal endothelial cells (Du et al., RNA toxicity and missplicing in the common eye disease fuchs endothelial corneal dystrophy. J Biol Chem. 2015 Mar. 6; 290(10):5979-90).

FECD represents a model disease for studying the biology of microsatellite disorders and evaluate novel therapeutic approaches. The unique anatomical position of the cornea provides access to diseased tissue and the relative immune privilege of cornea make it an ideal tissue for gene-based therapies while the therapeutic effect is easily monitored clinically.

Clustered regularly interspaced short palindromic repeats (CRISPR)/CRISPR associated Cas 9 nuclease are an extremely versatile and accurate approach to cut genomic DNA (Xu et al. (2015) Both TALENs and CRISPR/Cas9 directly target the HBB IVS2 654 (C>T) mutation in (3-thalassemia-derived iPSCs. Sci Rep., 9; 5:12065; Schwank et al. (2013) Functional repair of CFTR by CRISPR/Cas9 in intestinal stem cell organoids of cystic fibrosispatients. Cell Stem Cell; 13(6):653-8; Lee et al. (2015) T cells expressing CD19 chimeric antigen receptors for acute lymphoblastic leukaemia in children and young adults: a phase 1 dose-escalation trial. Lancet.; 385(9967):517-528). CRISPR/Cas9 can delete and replace genomic sequence using non-homologous end joining (NHEJ) or homology directed repair (HDR). NHEJ is the default repair pathway for double-stranded breaks (DSBs) and typically has higher frequency repair events than homology dependent repair (HDR) at all stages of the cell cycle. NHEJ repair typically results in disruption of the target site by indel formation, so is normally used for gene knockout studies, rather than repair of disease-causing mutations. However, NHEJ repair of two DSBs may result in target deletion of the genomic sequence between the two Cas9/gRNA target sites (Fujii et al., Efficient generation of large-scale genome-modified mice using gRNA and CAS9 endonuclease. Nucleic Acids Res. 2013 November; 41(20):e187).

### SUMMARY OF THE INVENTION

In one aspect, the present disclosure describes the potential of using CRISPR/Cas9 methods to simultaneously target Cas9 nuclease to sites flanking a disease-causing nucleotide repeat expansion. For example, the Cas9 targets both a site upstream and a site downstream of the nucleotide repeat expansion, with both target sites being located within the same intron between two protein-coding exons.

In one aspect, the present disclosure is related to an sgRNA pair designed for a CRISPR/Cas9 system. For example, the sgRNA pair may comprise (i) a first sgRNA comprising a first CRISPR targeting RNA (crRNA) sequence that hybridizes to a nucleotide sequence complementary to a first target sequence in a first intron, the first target sequence being positioned 5′ of a disease-causing repeat expansion that is present in the first intron, and (b) a tracrRNA sequence, in which the first crRNA sequence and the tracrRNA sequence do not naturally occur together; and (ii) a second sgRNA comprising (a) a second sgRNA comprising a second crRNA sequence that hybridizes to a nucleotide sequence complementary to a second target sequence in the first intron, the second target sequence being positioned 3′ of the disease-causing repeat expansion; (b) a tracrRNA sequence, in which the second crRNA sequence and the tracrRNA sequence do not naturally occur together. In some embodiments, the CRISPR/Cas9 system is for preventing, ameliorating or treating corneal dystrophies. In additional embodiments, the disease-causing repeat expansion is of the TCF4 gene. In further embodiments, at least one of the first and second crRNA sequences comprises a nucleotide sequence selected from the group consisting of guide sequences shown in FIG. 1A or in Table 1A (or Table 1A or Table 1B).

In one aspect, the present disclosure is related to an engineered Clustered Regularly Interspaced Short Palindromic Repeats (CRISPR)/CRISPR associate protein 9 (Cas9) system comprising at least one vector comprising a nucleotide molecule encoding Cas9 nuclease and the sgRNA pair described herein, wherein the Cas9 nuclease and said sgRNA pair in the vector do not naturally occur together.

In one aspect, the present disclosure is related to methods of preventing, ameliorating, or treating corneal dystrophy, the method comprising administering to the subject an engineered CRISPR/Cas9 system comprising at least one vector comprising at least two different CRISPR targeting RNA (crRNA) sequences or single guide RNA (sgRNA) sequences.

In one aspect, the present disclosure is related to a method of altering a gene product, the method comprising: administering into a cell an engineered CRISPR/Cas9 system comprising at least one vector comprising: (i) a nucleotide molecule encoding Cas9 nuclease; (ii) a first sgRNA comprising a first CRISPR targeting RNA (crRNA) sequence that hybridizes to a nucleotide sequence complementary to a first target sequence in a first intron, the first target sequence being positioned 5′ of a disease-causing repeat expansion that is present in the first intron; and (iii) a second sgRNA comprising a second crRNA sequence that hybridizes to a nucleotide sequence complementary to a second target sequence in the first intron, the second target sequence being positioned 3′ of the disease-causing repeat expansion, wherein the at least one vector does not have a nucleotide molecule encoding Cas9 nuclease and a crRNA sequence that naturally occur together.

In some embodiments, the first and second target sequences are positioned 5′ and 3′, respectively, of the intronic CTG18.1 trinucleotide repeat expansion of TCF4.

In some embodiments, at least one of the first and second crRNA sequences comprises a nucleotide sequence selected from the group consisting of guide sequences shown in FIG. 1A or in Table 1A (or Table 1A or Table 1B).

In some embodiments, the first crRNA sequence comprises the first target sequence; the second crRNA sequence comprises the second target sequence; the first crRNA sequence is from 17 to 24 nucleotide long; and/or the second crRNA sequence is from 17 to 24 nucleotide long.

In some embodiments, the first and/or second PAMs and the Cas9 nuclease are from Streptococcus or Staphylococcus.

In some embodiments, the first intron comprises a first protospacer adjacent motif (PAM). In some embodiments, the first PAM is adjacent to the first target sequence in the first intron. In some embodiments, the first PAM is next to the first target sequence in the first intron.

In some embodiments, the first intron comprises a second PAM. In some embodiments, the second PAM is adjacent to the second target sequence in the first intron. In some embodiments, the second PAM is next to the second target sequence in the first intron.

In some embodiments, the first and second PAMs are both from Streptococcus or Staphylococcus.

In some embodiments, each of the first and second PAMs independently consists of NGG or NNGRRT, wherein N is any of A, T, G, and C, and R is A or G.

In some embodiments, the administering comprises injecting the engineered CRISPR/Cas9 system into the cell.

In some embodiments, the administering comprises introducing the engineered CRISPR/Cas9 system into a cell containing and expressing a DNA molecule having the target sequence.

In some embodiments, the method includes administering the engineered CRISPR/Cas9 system into a subject.

In some embodiments, the subject is a human.

In some embodiments, disease is Fuchs' endothelial corneal dystrophy (FECD).

In some embodiments, the method further comprises, prior to administering to the subject the engineered CRISPR/Cas9 system: obtaining sequence information of the subject; and selecting the first crRNA sequence and/or the second crRNA sequence based on the sequence information of the subject.

In some embodiments, the sequence information of the subject includes whole-genome sequence information of the subject.

### BRIEF DESCRIPTION OF THE DRAWINGS

FIG. 1A-1B illustrates the design and screening of example gRNA pairs to target intronic trinucleotide repeat expansion in TCF4. Specifically, FIG. 1A shows a gene schematic diagram of TCF4 gene (SEQ ID NO: 47) indicating the position of the intronic CTG18.1 trinucleotide repeat locus, with the sequence marked in red, the guide sequences lying upstream and downstream of the repeat expansion underlined, and the PAM sites bolded, the table of FIG. 1A shows the sequences of Up1 (SEQ ID NO: 9), Up2 (SEQ ID NO: 10), Up3 (SEQ ID NO: 11), Up4 (SEQ ID NO: 12), Down1 (SEQ ID NO: 13), and Down2 (SEQ ID NO: 14) guides; and FIG. B shows in vitro digestion of DNA sequence with Cas9 Nuclease complexed with each of the sgRNA to assess activity of the guides. Gel lanes marked as T have the respective sgRNA added to them, and gel lanes marked as UD are controls having no sgRNA added; lane L is the 100 bp DNA ladder.

FIG. 2A-2B shows experimental results of TIDE analysis (Tracking Indels by Decomposition) that was carried out to assess genome editing efficiency by example sgRNAs in vitro. Specifically, FIG. 2A shows the frequency of indels assessed in the guides lying upstream of the TNR repeat; and FIG. 2B shows the frequency of indels assessed in the guides lying downstream of the TNR repeat.

FIG. 3 shows experimental results of PCR analysis to determine dual cut creating deletion of the trinucleotide repeat using example sgRNAs. The schematic representation shows the intron2 region of TCF4 with TNR with the vertical arrows showing the region where the sgRNAs cut. The horizontal arrows indicate the primer positions that were used to assess the deletion by dual sgRNA pairs. Gel lanes marked as T were treated with dual sgRNAs, and gel lanes marked as UT were untreated control. 100 ng of template DNA was used for PCR.

FIG. 4 shows experimental results of PCR analysis to determine deletion in cells using random oligos, with lane markers as follows: UT-untreated/untransfected, sg3 up-sg1dw—transfected with sg3 up—sg1dw RNP complex, sg3 up-sg1dw+R—transfected with sg3 up-sg1dw RNP complex and random single stranded oligo (Table 3A), sg4 up-sg1dw—transfected with sg4 up-sg1dw RNP complex, sg4 up-sg1dw+R—transfected with sg4 up-sg1dw RNP complex and random single stranded oligo.

FIG. 5A-5B shows experimental results of evaluation of efficiency of deletion frequency as measured by quantitative real time assay. Specifically, two qPCR amplifications, one with primers flanking the cut site of the sg3 up and sg1 down (FIG. 5A) and one with primers flaking the cut site of the sg4 up and sg1 down (FIG. 5B) were used to amplify and assess the efficiency of the dual cut across the deletion junction as shown in the schematic representations (top panels). The copy number was normalised with B-actin and that of an internal control EGFR gene (Epidermal Growth Factor Receptor). 2-way ANOVA was performed for statistical significance. ***<0.001, *<0.05. There was no significant difference in copy number of B-actin and EGFR genes in the treated and untreated cells.

### DETAILED DESCRIPTION OF THE INVENTION

As used throughout, ranges are used as shorthand for describing each and every value that is within the range. Any value within the range can be selected as the terminus of the range. In addition, all references cited herein are hereby incorporated by reference in their entireties for all purposes. In the event of a conflict in a definition in the present disclosure and that of a cited reference, the present disclosure controls.

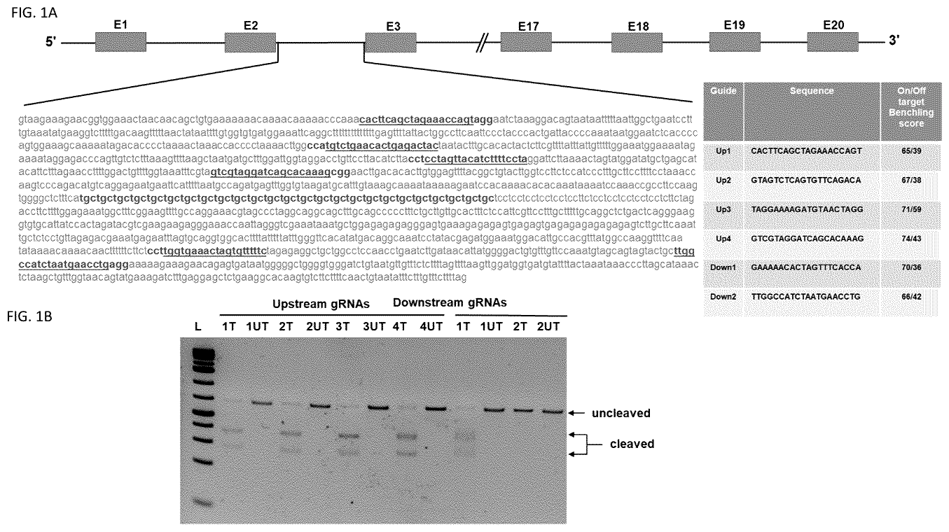

In one aspect, the present disclosure is related to an sgRNA pair designed for a CRISPR/Cas9 system. For example, the sgRNA pair may comprise (i) a first sgRNA comprising (a) a first sgRNA comprising a first CRISPR targeting RNA (crRNA) sequence that hybridizes to a nucleotide sequence complementary to a first target sequence in a first intron, the first target sequence being positioned 5′ of a disease-causing repeat expansion that is present in the first intron, and (b) a tracrRNA sequence, in which the first crRNA sequence and the tracrRNA sequence do not naturally occur together; and (ii) a second sgRNA comprising (a) a second sgRNA comprising a second crRNA sequence that hybridizes to a nucleotide sequence complementary to a second target sequence in the first intron, the second target sequence being positioned 3′ of the disease-causing repeat expansion; (b) a tracrRNA sequence, in which the second crRNA sequence and the tracrRNA sequence do not naturally occur together. In some embodiments, the CRISPR/Cas9 system is for preventing, ameliorating or treating the diseases disclosed herein. In some embodiments, the corneal dystrophy is associated with an intronic CTG18.1 trinucleotide repeat expansion of TCF4. In further embodiments, at least one of the first and second crRNA sequences comprises a nucleotide sequence selected from the group consisting of guide sequences shown in FIG. 1A or Table 1A (or Table 1A or Table 1).

The term “crRNA” may refer to a guide sequence that may be a part of an sgRNA in an CRISPR/Cas9 system. In some embodiments, at least one of the first and second crRNA sequences described herein comprises a nucleotide sequence selected from the group consisting of sequences listed in FIG. 1A; and/or at least one of the first and second crRNA sequences comprises a nucleotide sequence selected from the group consisting of sequences listed in Table 1A (or Table 1A or Table 1B). The term, “sgRNA” refers to a single guide RNA containing (i) a guide sequence (crRNA sequence) and (ii) a Cas9 nuclease-recruiting sequence (tracrRNA). The crRNA sequence may be a sequence that is homologous to a region in your gene of interest and may direct Cas9 nuclease activity. The crRNA sequence and tracrRNA sequence may not naturally occur together. The sgRNA may be delivered as RNA or by transforming with a plasmid with the sgRNA-coding sequence (sgRNA gene) under a promoter. The tracrRNA sequence may be any sequence for tracrRNA for CRISPR/Cas9 system known in the art.

As used herein, a “corneal dystrophy” refers to any one of a group of hereditary disorders in the outer layer of the eye (cornea). For example, the corneal dystrophy may be characterized by bilateral abnormal deposition of substances in the cornea. Corneal dystrophies include, but are not limited to the following four IC3D categories of corneal dystrophies (see, e.g., Weiss et al., Cornea 34(2): 117-59 (2015)): epithelial and sub-epithelial dystrophies, epithelial-stromal TGFβI dystrophies, stromal dystrophies and endothelial dystrophies. In some embodiments, the corneal dystrophy is Fuchs' endothelial corneal dystrophy (FECD). In some embodiments, the FECD is associated with an intronic CTG18.1 trinucleotide repeat expansion of Transcription factor 4 (TCF4).

In some embodiments, the crRNA hybridizes to at least a part of a target sequence (e.g., target genome sequence), and the crRNA may have a complementary sequence to the target sequence. In some embodiments, the crRNA may comprise the first target sequence or the second target sequence. In additional embodiments, the first and second target sequences are located in introns of a target gene. “Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least about 60%, 65%, 70%, 75%, 80%, 85%, 90%, 95%. 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part 1, Second Chapter “Overview of principles of hybridization and the strategy of nucleic acid probe assay”, Elsevier, N.Y. “Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence. In additional embodiments, the crRNA or the guide sequence is about 17, 18, 19, 20, 21, 22, 23 or 24 nucleotide long. As used herein, the term “about” may refer to a range of values that are similar to the stated reference value. In certain embodiments, the term “about” refers to a range of values that fall within 15, 10, 9, 8, 7, 6, 5, 4, 3, 2, 1 percent or less of the stated reference value.

In one aspect, the present disclosure is related to methods of preventing, ameliorating, or treating a disease associated with a repeat expansion in a subject, comprising administering to the subject an engineered CRISPR/Cas9 system comprising at least one vector comprising (i) a nucleotide molecule encoding Cas9 nuclease; (ii) a first sgRNA comprising a first CRISPR targeting RNA (crRNA) sequence that hybridizes to a nucleotide sequence complementary to a first target sequence in a first intron, the first target sequence being positioned 5′ of a disease-causing repeat expansion that is present in the first intron; and (iii) a second sgRNA comprising a second crRNA sequence that hybridizes to a nucleotide sequence complementary to a second target sequence in the first intron, the second target sequence being positioned 3′ of the disease-causing repeat expansion, wherein at least one vector does not have a nucleotide molecule encoding Cas9 nuclease and a crRNA sequence that naturally occur together. In another aspect, the present disclosure is related to methods of preventing, ameliorating, or treating a disease associated with a repeat expansion in a subject comprising altering expression of the gene product of the subject by the methods described above, wherein the gene comprises a repeat expansion sequence. In some embodiments, the subject is human. In some embodiments, the first crRNA sequence hybridizes to the nucleotide sequence so that the Cas9 nuclease cleaves at a first cleaving site that is positioned 5′ of the disease-causing repeat expansion in the first intron; and the second crRNA sequence hybridizes to the nucleotide sequence so that the Cas9 nuclease cleaves at a second cleaving site that is positioned 3′ of the disease-causing repeat expansion in the first intron.

In some embodiments, the engineered CRISPR/Cas9 system described herein may comprise at least one vector comprising (i) a nucleotide molecule encoding Cas9 nuclease described herein, and (ii) a plurality of sgRNA targeting intronic sites flanking one or more disease-causing repeat expansions of interest as described herein. In some embodiments, the Cas9 nuclease and the sgRNA do not naturally occur together. In yet additional embodiments, the PAM consists of a PAM selected from the group consisting of NGG and NNGRRT, wherein N is any of A, T, G, and C, and R is A or G.

In some embodiments, the disease-causing repeat expansion is in an intron of a gene associated with the disease, and the first and second target sequences are located within the same intron, positioned 5′ and 3′ of the repeat expansion, respectively. In some embodiments, the disease-causing repeat expansion is in an exon of a gene associated with the disease, and the first and second target sequences are located within the same exon, positioned 5′ and 3′ of the repeat expansion, respectively. In some embodiments, first and second CRISPR targeting RNA (crRNA) sequences hybridize to nucleotide sequences complementary to first and second target sequences, the first target sequence being positioned 5′ of the repeat expansion, and the second target sequence being positioned 3′ of the repeat expansion. Thus, the Cas9 cleaves at sites flanking the repeat expansion, causing deletion of the repeat expansion.

As used herein, an “intron” means a section of DNA occurring between two adjacent exons within a gene which is removed during pre-mRNA splicing and does not code for any amino acids constituting the gene product. An “intronic site” is a site within an intron. An “exon” means a section of DNA occurring in a gene which codes for one or more amino acids in the gene productAn “exonic site” is a site within an exon.

As used herein, a “repeat expansion” means a mutant nucleic acid molecule having a nucleobase sequence that includes a repeat region having a number of nucleobase repeats, wherein the presence or length of the repeat region affects the normal processing, function, or activity of the RNA or corresponding protein. “Repeat expansions” are also referred to as “microsatellite repeats” or “nucleotide repeat expansions.”

In some embodiments, the first crRNA sequence comprises the first target sequence, and the second crRNA sequence comprises the second target sequence. In further embodiments, each of the first crRNA sequence and the second crRNA sequence may independent be from 14, 15, 16, 17, 18, 19, 20, 21, 22, 23, 24 or 25 to 20, 21, 22, 23, 24, 25, 26, 27, 28, 29 or 30 nucleotide long.

In some embodiments, the methods described herein further comprise identifying targetable sites on either side of disease-causing repeat expansions. In some embodiments, a block of DNA is identified in a phased sequencing experiment. In some embodiments, identifying sites on both side of the disease-causing repeat expansions that are suitable for CRISPR/Cas9 cleavage allows removal of a segment of DNA that includes the disease-causing repeat expansions. In some embodiments, the read length may be increased so as to gain longer contiguous reads and a haplotype phased genome by using a technology described in Weisenfeld N I, Kumar V, Shah P, Church D M, Jaffe D B. Direct determination of diploid genome sequences. Genome research. 2017; 27(5):757-767, which is herein incorporated by reference in its entirety.

In some embodiments, the methods described herein further comprises, prior to administering to the subject the engineered CRISPR/Cas9 system, obtaining genomic or sequence information of the subject; and selecting the first crRNA sequence and/or the second crRNA sequence based on the genomic or sequence information of the subject. In additional embodiments, the genomic or sequence information of the subject includes whole or partial genome sequence information of the subject.

The human genome is diploid by nature; every chromosome with the exception of the X and Y chromosomes in males is inherited as a pair, one from the male and one from the female parent. When seeking stretches of contiguous DNA sequence larger than a few thousand base pairs, a determination of inheritance is crucial to understand from which parent these blocks of DNA originate. Longer read sequencing technologies have been utilized in attempts to produce a haplotype-resolved genome sequences, i.e. haplotype phasing. Thus, when investigating the genomic sequence of a particular stretch of DNA longer than 50 kbps, a haplotype phased sequence analysis may be utilized to determine which of the paired chromosomes carries the sequence of interest. Longer phased sequencing reads may be employed to determine whether the disease-causing repeat expansion of interest would be suitable as a target for the CRISPR/Cas9 gene editing system described herein.

In some embodiments, the selected first crRNA sequence is configured to cause cleaving at a first cleaving site, within genome of the subject, that is 5′ of a disease-causing repeat expansion; and the selected second crRNA sequence is configured to cause cleaving at a second cleaving site, within the genome of the subject, that is 3′ of a disease-causing repeat expansion.

In some embodiments, the subjects that can be treated with the methods described herein include, but are not limited to, mammalian subjects such as a mouse, rat, dog, baboon, pig or human. In some embodiments, the subject is a human. The methods can be used to treat subjects at least 1 year, 2 years, 3 years, 5 years, 10 years, 15 years, 20 years, 25 years, 30 years, 35 years, 40 years, 45 years, 50 years, 55 years, 60 years, 65 years, 70 years, 75 years, 80 years, 85 years, 90 years, 95 years or 100 years of age. In some embodiments, the subject is treated for at least one, two, three, or four diseases. For example, a single or multiple crRNA or sgRNA may be designed to alter or delete nucleotides at more than 2, 3, 4, 5, 6, 7, 8, 9 or 10 and/or fewer than 20, 10, 9, 8, 7, 6, 5, 4 or 3 repeat expansion sites.

In some embodiments, the methods of preventing, ameliorating, or treating the disease in a subject may comprise administering to the subject an effective amount of the engineered CRISPR/Cas9 system described herein. The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

In some embodiments, the administering comprises injecting the engineered CRISPR/Cas9 system into the subject. In additional embodiments, the administering comprises introducing the engineered CRISPR/Cas9 system into a cell containing and expressing a DNA molecule having the target sequence as described below.

In some embodiments, the methods of treating the disease provide a positive therapeutic response with respect to a disease or condition. By “positive therapeutic response” is intended an improvement in the disease or condition, and/or an improvement in the symptoms associated with the disease or condition. The therapeutic effects of the subject methods of treatment can be assessed using any suitable method. In some embodiments, the subject methods reduce the amount of a disease-associate protein deposition in the subject by at least 5%, 10%, 15%, 20%, 25%, 30%, 35%, 40%, 45%, 50%, 55%, 60%, 65%, 70%, 75%, 80%, 85%, 90%, 95%, or 99% as compared to the subject prior to undergoing treatment.

In another aspect, the present disclosure is related to engineered Clustered Regularly Interspaced Short Palindromic Repeats (CRISPR)/CRISPR associate protein 9 (Cas9) systems for preventing, ameliorating or treating corneal dystrophies. The CRISPR/Cas9 may comprise at least one vector comprising a nucleotide molecule encoding Cas9 nuclease and the sgRNAs and/or crRNAs as described herein. The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature. In some embodiments, the Cas9 nuclease and the sgRNA/crRNA do not naturally occur together.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as “crRNA” herein, or a “spacer” in the context of an endogenous CRISPR system), and/or other sequences and transcripts from a CRISPR locus. As described above, sgRNA is a combination of at least tracrRNA and crRNA. In some embodiments, one or more elements of a CRISPR system are derived from a type II CRISPR system. In some embodiments, one or more elements of a CRISPR system are derived from a particular organism comprising an endogenous CRISPR system, such as Streptococcus pyogenes or Staphylococcus aureus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” may refer to a sequence to which a guide sequence is designed to have complementarity, where hybridization between a target sequence and a guide sequence promotes the formation of a CRISPR complex, the “target sequence” may refer to a sequence adjacent to a PAM site, which the guide sequence comprises. Full complementarity is not necessarily required, provided there is sufficient complementarity to cause hybridization and promote formation of a CRISPR complex. In this disclosure, “target site” refers to a site of the target sequence including both the target sequence and its complementary sequence, for example, in double stranded nucleotides. In some embodiments, the target site described herein may mean a first target sequence hybridizing to sgRNA or crRNA of CRISPR/Cas9 system, and/or a second target sequence adjacent to the 5′-end of a PAM. A target sequence may comprise any polynucleotide, such as DNA or RNA polynucleotides. In some embodiments, a target sequence is located in the nucleus or cytoplasm of a cell. In some embodiments, the target sequence may be within an organelle of a eukaryotic cell, for example, mitochondrion or chloroplast.

The term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids. Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells.

In some embodiments, at least one vector of the engineered CRISPR/Cas9 system described herein further comprises (a) a first regulatory element operably linked to the sgRNA that hybridizes with the target sequence described herein, and (b) a second regulatory element operably linked to the nucleotide molecule encoding Cas9 nuclease, wherein components (a) and (b) are located on a same vector or different vectors of the system, the sgRNA targets the target sequence, and the Cas9 nuclease cleaves the DNA molecule. The target sequence may be a nucleotide sequence complementary to from 16 to 25 nucleotides adjacent to the 5′ end of a PAM. Being “adjacent” herein means being within 2 or 3 nucleotides of the site of reference, including being “immediately adjacent,” which means that there is no intervening nucleotides between the immediately adjacent nucleotide sequences and the immediate adjacent nucleotide sequences are within 1 nucleotide of each other. In additional embodiments, the cell is a eukaryotic cell, or a mammalian or human cell, and the regulatory elements are eukaryotic regulators. In further embodiments, the cell is a stem cell described herein. In some embodiments, the Cas9 nuclease is codon-optimized for expression in a eukaryotic cell.

In some embodiments, the first regulatory element is a polymerase III promoter. In some embodiments, the second regulatory element is a polymerase II promoter. The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol I promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the 3-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA, Vol. 78(3), p. 1527-31, 1981).

In some embodiments, the Cas9 nuclease provided herein may be an inducible Cas9 nuclease that is optimized for expression in a temporal or cell-type dependent manner. The first regulatory element may be an inducible promoter that can be linked to the Cas9 nuclease include, but are not limited to, tetracycline-inducible promoters, metallothionein promoters; tetracycline-inducible promoters, methionine-inducible promoters (e.g., MET25, MET3 promoters); and galactose-inducible promoters (GAL1, GAL7 and GAL10 promoters). Other suitable promoters include the ADH1 and ADH2 alcohol dehydrogenase promoters (repressed in glucose, induced when glucose is exhausted and ethanol is made), the CUP1 metallothionein promoter (induced in the presence of Cu²⁺, Zn²⁺), the PHO5 promoter, the CYC1 promoter, the HIS3 promoter, the PGK promoter, the GAPDH promoter, the ADC1 promoter, the TRP1 promoter, the URA3 promoter, the LEU2 promoter, the ENO promoter, the TP1 promoter, and the AOX1 promoter.

It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

Exemplary CRISPR/Cas9 systems, sgRNA, crRNA and tracrRNA, and their manufacturing process and use are disclosed in U.S. Pat. No. 8,697,359, U.S. Patent Application Publication Nos. 20150232882, 20150203872, 20150184139, 20150079681, 20150073041, 20150056705, 20150031134, 20150020223, 20140357530, 20140335620, 20140310830, 20140273234, 20140273232, 20140273231, 20140256046, 20140248702, 20140242700, 20140242699, 20140242664, 20140234972, 20140227787, 20140189896, 20140186958, 20140186919, 20140186843, 20140179770, 20140179006, 20140170753, 20140093913, 20140080216, and WO2016049024, all of which are incorporated herein by their entirety.

In some embodiments, the Cas9 nucleases described herein are known; for example, the amino acid sequence of S. pyogenes Cas9 protein may be found in the SwissProt database under accession number Q99ZW2. The Cas9 nuclease may be a Cas9 homolog or ortholog. Mutant Cas9 nucleases that exhibit improved specificity may also be used (see, e.g., Ann Ran et al. Cell 154(6) 1380-89 (2013), which is herein incorporated by reference in its entirety for all purposes and particularly for all teachings relating to mutant Cas9 nucleases with improved specificity for target nucleic acids). The nucleic acid manipulation reagents can also include a deactivated Cas9 nuclease (dCas9). Deactivated Cas9 binding to nucleic acid elements alone may repress transcription by sterically hindering RNA polymerase machinery. Further, deactivated Cas may be used as a homing device for other proteins (e.g., transcriptional repressor, activators and recruitment domains) that affect gene expression at the target site without introducing irreversible mutations to the target nucleic acid. For example, dCas9 can be fused to transcription repressor domains such as KRAB or SID effectors to promote epigenetic silencing at a target site. Cas9 can also be converted into a synthetic transcriptional activator by fusion to VP16/VP64 or p64 activation domains. In some instances, a mutant Type II nuclease, referred to as an enhanced Cas9 (eCa9) nuclease, is used in place of the wild-type Cas9 nuclease. The enhanced Cas9 has been rationally engineered to improve specificity by weakening non-target binding. This has been achieved by neutralizing positively charged residues within the non-target strand groove (Slaymaker et al., 2016).

In some embodiments, the Cas9 nucleases direct cleavage of one or both strands at the location of a target sequence, such as within the target sequence and/or within the complement of the target sequence. In some embodiments, the Cas9 nucleases directs cleavage of one or both strands within about 1, 2, 3, 4, 5, 6, 7, 8, 9, 10, 15, 20, 25, 50, 100, 200, 500, or more base pairs from the first or last nucleotide of a target sequence.

Following directed DNA cleavage by the Cas9 nuclease, there are two modes of DNA repair available to the cell: homology directed repair (HDR) and non-homologous end joining (NHEJ). While seamless correction of the mutation by HDR following Cas9 cleavage close to the mutation site is attractive, the efficiency of this method means that it could only be used for in vitro/ex vivo modification of stem cells or induced pluripotent stem cells (iPSC) with an additional step to select those cells in which repair had taken place and purify those modified cells only. HDR does not occur at a high frequency in cells.

In some embodiments, the first and/or second PAMs and the Cas9 nuclease described herein are from Streptococcus or Staphylococcus. In additional embodiments, the first and second PAMs are both from Streptococcus or Staphylococcus. In additional embodiments, the Cas9 nuclease is from Streptococcus. In yet additional embodiments, the Cas9 nuclease is from Streptococcus pyogenes, Streptococcus dysgalactiae, Streptococcus canis, Streptococcus equi, Streptococcus iniae, Streptococcus phocae, Streptococcus pseudoporcinus, Streptococcus oralis, Streptococcus pseudoporcinus, Streptococcus infantarius, Streptococcus mutans, Streptococcus agalactiae, Streptococcus caballi, Streptococcus equinus, Streptococcus sp. oral taxon, Streptococcus mitis, Streptococcus gallolyticus, Streptococcus gordonii, or Streptococcus pasteurianus, or variants thereof. Such variants may include D10A Nickase, Spy Cas9-HF1 as described in Kleinstiver et al, 2016 Nature, 529, 490-495, or Spy eCas9 as described in Slaymaker et al., 2016 Science, 351(6268), 84-88. In additional embodiments, the Cas9 nuclease is from Staphylococcus. In yet additional embodiments, the Cas9 nuclease is from Staphylococcus aureus, S. simiae, S. auricularis, S. carnosus, S. condimenti, S. massiliensis, S. piscifermentans, S. simulans, S. capitis, S. caprae, S. epidermidis, S. saccharolyticus, S. devriesei, S. haemolyticus, S. hominis, S. agnetis, S. chromogenes, S. felis, S. delphini, S. hyicus, S. intermedius, S. lutrae, S. microti, S. muscae, S. pseudintermedius, S. rostri, S. schleiferi, S. lugdunensis, S. arlettae, S. cohnii, S. equorum, S. gallinarum, S. kloosii, S. leei, S. nepalensis, S. saprophyticus, S. succinus, S. xylosus, S. fleurettii, S. lentus, S. sciuri, S. stepanovicii, S. vitulinus, S. simulans, S. pasteuri, S. warneri, or variants thereof.

In further embodiments, the Cas9 nuclease excludes Cas9 nuclease from Streptococcus pyogenes.

In additional embodiments, the Cas9 nuclease comprises an amino acid sequence having at least about 60, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with an amino acid sequence selected from the group consisting of SEQ ID NO: 4 or 8. In additional embodiments, the nucleotide molecule encoding Cas9 nuclease comprises a nucleotide sequence having at least about 60, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with a nucleotide sequence selected from the group consisting of SEQ ID NO: 3 or 7. In yet additional embodiments, Cas9 sgRNA sequence may comprises a sequence having at least about 60, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO: 1 or 5. An exemplary tracrRNA or sgRNA scaffold sequence may comprise a sequence having at least about 60, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO: 2 or 6.

In some embodiments, the Cas9 nuclease is an enhanced Cas9 nuclease that has one or more mutations improving specificity of the Cas9 nuclease. In additional embodiments, the enhanced Cas9 nuclease is from a Cas9 nuclease from Streptococcus pyogenes having one or more mutations neutralizing a positively charged groove, positioned between the HNH, RuvC, and PAM-interacting domains in the Cas9 nuclease. In yet additional embodiments, the Cas9 nuclease comprises an amino acid sequence having at least about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with a mutant amino acid sequence of a Cas9 nuclease from Streptococcus pyogenes (e.g., SEQ ID NO: 4) with one or more mutations selected from the group consisting of (i) K855A, (ii) K810A, K1003A and R1060A, and (iii) K848A, K1003A and R1060A. In yet further embodiments, the nucleotide molecule encoding Cas9 nuclease comprises a nucleotide sequence having at least about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with a nucleotide sequence encoding the mutant amino acid sequence.

In some embodiments, the CRISPR/Cas9 system and the methods using the CRISPR/Cas9 system described herein alter a DNA sequence by the NHEJ. In additional embodiments, the CRISPR/Cas9 system or the vector described herein does not include a repair nucleotide molecule. In some embodiments, the methods described herein alter a DNA sequence by the HDR. In some embodiments, the CRISPR/Cas9 system or the vector described herein may further comprise a repair nucleotide molecule. The target polynucleotide cleaved by the Cas9 nuclease may be repaired by homologous recombination with the repair nucleotide molecule, which is an exogenous template polynucleotide. This repair may result in a mutation comprising an insertion, deletion, or substitution of one or more nucleotides of said target polynucleotide. The repair nucleotide molecule introduces a specific allele (e.g., a wild-type allele) into the genome of one or more cells of the plurality of stem cells upon repair of a Type II nuclease induced DSB through the HDR pathway. In some embodiments, the repair nucleotide molecule is a single stranded DNA (ssDNA). In other embodiments, the repair nucleotide molecule is introduced into the cell as a plasmid vector. In some embodiments, the repair nucleotide molecule is 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 105, 105 to 110, 110 to 115, 115 to 120, 120 to 125, 125 to 130, 130 to 135, 135 to 140, 140 to 145, 145 to 150, 150 to 155, 155 to 160, 160 to 165, 165 to 170, 170 to 175, 175 to 180, 180 to 185, 185 to 190, 190 to 195, or 195 to 200 nucleotides in length. In some embodiments, the repair nucleotide molecule is 200 to 300, 300, to 400, 400 to 500, 500 to 600, 600 to 700, 700 to 800, 800 to 900, 900 to 1,000 nucleotides in length. In other embodiments, the repair nucleotide molecule is 1,000 to 2,000, 2,000 to 3,000, 3,000 to 4,000, 4,000 to 5,000, 5,000 to 6,000, 6,000 to 7,000, 7,000 to 8,000, 8,000 to 9,000, or 9,000 to 10,000 nucleotides in length.

The repair nucleotide molecule may further include a label for identification and sorting of cells described herein containing the specific mutation. Exemplary labels that can be included with the repair nucleotide molecule include fluorescent labels and nucleic acid barcodes that are identifiable by length or sequence.

In additional embodiments, the CRISPR/Cas9 system or the vector described herein may include at least one nuclear localization signal (NLS). In additional embodiments, the sgRNA and the Cas9 nuclease are included on the same vector or on different vectors.

In another aspect, the present disclosure is also related to methods of altering expression of at least one gene product comprising introducing the engineered CRISPR/Cas9 system described herein into a cell containing and expressing a DNA molecule having a target sequence and encoding the gene product. The engineered CRISPR/Cas9 system can be introduced into cells using any suitable method. In some embodiments, the introducing may comprise administering the engineered CRISPR/Cas9 system described herein to cells in culture, or in a host organism.

Exemplary methods for introducing the engineered CRISPR/Cas9 system include, but are not limited to, transfection, electroporation and viral-based methods. In some cases, the one or more cell uptake reagents are transfection reagents. Transfection reagents include, for example, polymer based (e.g., DEAE dextran) transfection reagents and cationic liposome-mediated transfection reagents. Electroporation methods may also be used to facilitate uptake of the nucleic acid manipulation reagents. By applying an external field, an altered transmembrane potential in a cell is induced, and when the transmembrane potential net value (the sum of the applied and the resting potential difference) is larger than a threshold, transient permeation structures are generated in the membrane and electroporation is achieved. See, e.g., Gehl et al., Acta Physiol. Scand. 177:437-447 (2003). The engineered CRISPR/Cas9 system also may be delivered through viral transduction into the cells. Suitable viral delivery systems include, but are not limited to, adeno-associated virus (AAV), retroviral and lentivirus delivery systems. Such viral delivery systems are useful in instances where the cell is resistant to transfection. Methods that use a viral-mediated delivery system may further include a step of preparing viral vectors encoding the nucleic acid manipulation reagents and packaging of the vectors into viral particles. Other method of delivery of nucleic acid reagents include, but are not limited to, lipofection, nucleofection, microinjection, biolistics, virosomes, liposomes, immunoliposomes, polycation or lipid:nucleic acid conjugates, naked DNA, artificial virions, and agent-enhanced uptake of nucleic acids. See, also Neiwoehner et al., Nucleic Acids Res. 42:1341-1353 (2014), and U.S. Pat. Nos. 5,049,386, 4,946,787; and 4,897,355, which are herein incorporated by reference in its entirety for all purposes, and particularly for all teachings relating to reagent delivery systems. In some embodiments, the introduction is performed by non-viral vector delivery systems include DNA plasmids, RNA (e.g., a transcript of a vector described herein), naked nucleic acid, and nucleic acid complexed with a delivery vehicle, such as a liposome. Delivery can be to cells (e.g., in vitro or ex vivo administration) or target tissues (e.g., in vivo administration). The cells that have undergone a nucleic acid alteration event (i.e., a “altered” cell) can be isolated using any suitable method. In some embodiments, the repair nucleotide molecule further comprises a nucleic acid encoding a selectable marker. In these embodiments, successful homologous recombination of the repair nucleotide molecule a host stem cell genome is also accompanied by integration of the selectable marker. Thus, in such embodiments, the positive marker is used to select for altered cells. In some embodiments, the selectable marker allows the altered cell to survive in the presence of a drug that otherwise would kill the cell. Such selectable markers include, but are not limited to, positive selectable markers that confer resistance to neomycin, puromycin or hygromycin B. In addition, a selectable marker can be a product that allows an altered cell to be identified visually among a population of cells of the same type, some of which do not contain the selectable marker. Examples of such selectable markers include, but are not limited to the green fluorescent protein (GFP), which can be visualized by its fluorescence; the luciferase gene, which, when exposed to its substrate luciferin, can be visualized by its luminescence; and β-galactosidase (β-gal), which, when contacted with its substrate, produces a characteristic color. Such selectable markers are well known in the art and the nucleic acid sequences encoding these markers are commercially available (see, e.g., Sambrook et al., Molecular Cloning: A Laboratory Manual (Cold Spring Harbor Laboratory Press 1989). Methods that employ selectable markers that can be visualized by fluorescence may further be sorted using Fluorescence Activated Cell Sorting (FACS) techniques. Isolated manipulated cells may be used to establish cell lines for transplantation. The isolated altered cells can be cultured using any suitable method to produce a stable cell line.

In another aspect, the present disclosure is related to methods of treating a disease associated with a repeat expansion in a subject in need thereof, comprising: (a) obtaining a plurality of stem cells comprising a repeat expansion in a corneal dystrophy target nucleic acid from the subject; (b) manipulating the repeat expansion in one or more stem cells of the plurality of stem cells to delete the repeat expansion, thereby forming one or more manipulated stem cells; (c) isolating the one or more manipulated stem cells; and (d) transplanting the one or more manipulated stem cells into the subject, wherein manipulating the nucleic acid mutation in the one or more stem cells of the plurality of stem cells includes performing any of the methods of altering expression of a gene product or of preventing, ameliorating, or treating a disease associated with a repeat expansion in a subject as described herein.

The subject methods may include obtaining a plurality of stem cells. Any suitable stem cells can be used for the subject method, depending on the type of the disease to be treated. In certain embodiments, the stem cell is obtained from a heterologous donor. In such embodiments, the stem cells of the heterologous donor and the subject to be treated are donor-recipient histocompatible. In certain embodiments, autologous stem cells are obtained from the subject in need of the treatment for the disease. Obtained stem cells carry a mutation in a gene associated with the particular disease to be treated. Suitable stem cells include, but are not limited to, dental pulp stem cells, hair follicle stem cells, mesenchymal stem cells, umbilical cord lining stem cells, embryonic stem cells, oral mucosal epithelial stem cells and limbal epithelial stem cells.

Stem cells to be manipulated may include individual isolated stem cells or stem cells from a stem cell line established from the isolated stem cells. Any suitable genetic manipulation method may be used to correct the nucleic acid mutation in the stem cells.

In another aspect, provided herein are kits comprising the CRISPR/Cas9 system for the treatment of a disease associated with a repeat expansion. In some embodiments, the kit includes one or more sgRNAs described herein, and a Cas9 nuclease as described herein. In some embodiments, the kit also includes agents that facilitate uptake of the nucleic acid manipulation by cells, for example, a transfection agent or an electroporation buffer. In some embodiments, the subject kits provided herein include one or more reagents for the detection or isolation of stem cells, for example, labeled antibodies for one or more positive stem cell markers that can be used in conjunction with FACS.

In another aspect, the present disclosure is related to an sgRNA pair, and a kit comprising the sgRNA pair comprising at least two sgRNAs for CRISPR/Cas9 system to delete a disease-causing repeat expansion, for example, for preventing, ameliorating or treating corneal dystrophies. In some embodiments, the sgRNA pair comprises an sgRNA comprising a first sgRNA comprising a first CRISPR targeting RNA (crRNA) sequence that hybridizes to a nucleotide sequence complementary to a first target sequence in a first intron, the first target sequence being positioned 5′ of a disease-causing repeat expansion that is present in the first intron, and a second sgRNA comprising a second crRNA sequence that hybridizes to a nucleotide sequence complementary to a second target sequence in the first intron, the second target sequence being positioned 3′ of the disease-causing repeat expansion.

### EXAMPLES

The following examples are presented to illustrate various embodiments of the invention. It is understood that such examples do not represent and are not intended to represent exclusive embodiments; such examples serve merely to illustrate the practice of this invention.

### Example 1. Design and Screening of gRNA Pairs to Target Intronic Trinucleotide Repeat Expansion in TCF4

The CRISPR design tool identified several target sites (gRNA sequences) lying upstream and downstream of the trinucleotide repeat expansion in intron 2 of TCF4. The identified gRNAs were ranked by the best combination for the on-target and off-target activity scores and the top 4 gRNAs lying upstream of the TNR and top 2 gRNAs lying downstream of the TNR were selected for further experiments (FIG. 1A). To confirm the activity of the guides, an in vitro digestion using the sgRNAs with a reporter containing the target sequence was carried out (FIG. 1B). All the upstream sgRNAs guided the Cas9 nuclease to cleave the target sequences efficiently. However, only the sgRNA1 of the downstream guides cleaved the target in vitro (FIG. 1B).

### Example 2. TIDE (Tracking Indels by Decomposition) Analysis to Assess Genome Editing Efficiency

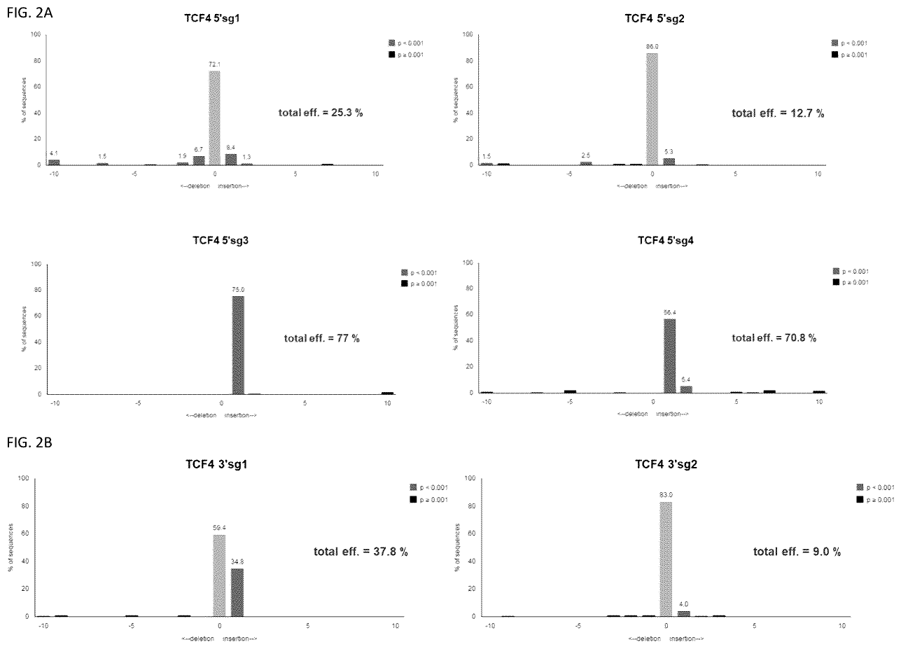

To assess the activity of the sgRNAs in cells in vitro, HEK293 cells were transfected with ribonucleoprotein (RNP) complexes consisting of Cas9 nuclease complexed with each synthetic single guide RNA in turn. Genomic DNA extracted from the transfected HEK293 cells after 72 hours was amplified by PCR using primers flanking the target site, sequenced by the dideoxy chain termination method, and the resultant sequence was analysed by TIDE to access the frequency of indels generated by non-homologous end joining repair (NHEJ). The indel frequency for the upstream sgRNAs was determined to be sgRNA1—25.3%, sgRNA2-12.7%, sgRNA3—77% and sgRNA4—70.8% respectively (FIG. 2A). The downstream sgRNA1 and sgRNA2 had frequencies of 37.8% and 9% respectively (FIG. 2B). This confirms the genome editing ability of the guide RNAs in in vitro cells. The upstream sgRNA3 and sgRNA4 exhibited high editing frequencies. The downstream sgRNA1 showed moderate frequency whereas downstream sgRNA2 had low frequency. The indel frequency assessed using TIDE analysis only represents the fraction of the Cas9/sgRNA cuts which are not seamlessly repaired.

### Example 3. TNR Deletion by Dual Cut Approach in In Vitro

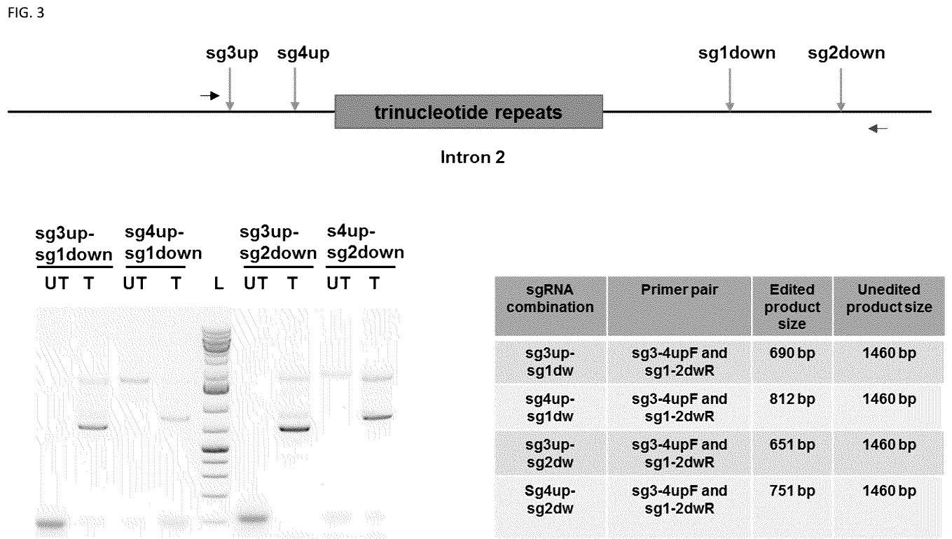

The upstream (sgRNA3 and sgRNA4) and downstream (sgRNA1) guides exhibiting the highest gene editing frequencies were tested, in pairs, for their ability to delete the trinucleotide repeat in intron 2 of TCF4. HEK293 cells were transfected with the different combinations of RNP complexes, and extracted genomic DNA was analysed to detect the predicted deletion. Transfection with both the different combinations of RNP complexes (sg3 up-sg1 down, sg4 up-sg1 down) resulted in a deletion of the trinucleotide repeat. PCR analysis, using primers flanking the predicted deletion, demonstrated amplification of shorter fragments in the treated samples compared to the larger fragments seen in the untreated samples and thus showed the TNR deletion by dual cut approach worked (FIG. 3). The deletion PCR for all the combinations showed similar efficiency comparing the band intensities (FIG. 3).

Analysis of Random Oligos

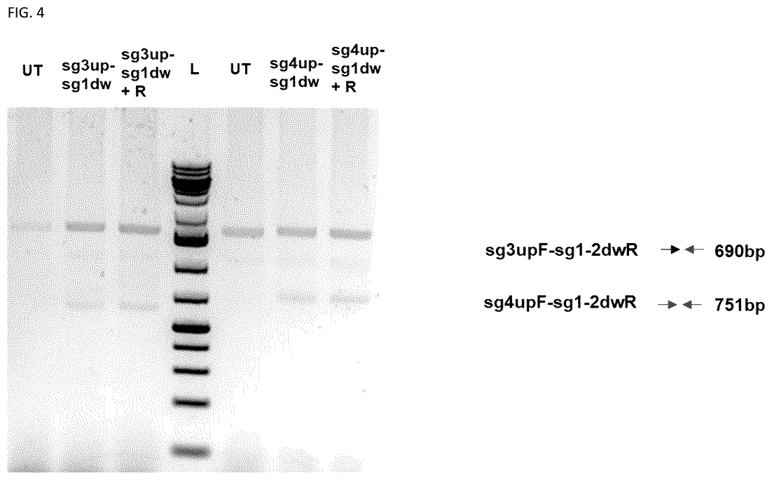

To investigate whether it is possible to increase the efficiency of TNR excision by the sgRNAs, BEK 293 cells were co-transfected with the combination of sg3 up-sg1 down and sg4 up-sg1 down RNP complex and a random non-homologous single stranded DNA. By comparing the band intensities of PCR products, no increase in efficiency of deletion was observed in cells co-transfected with random non-homologous single stranded DNA when compared with the cells not treated with the random oligo (FIG. 4).

Efficiency of Targeted Excision of TNR

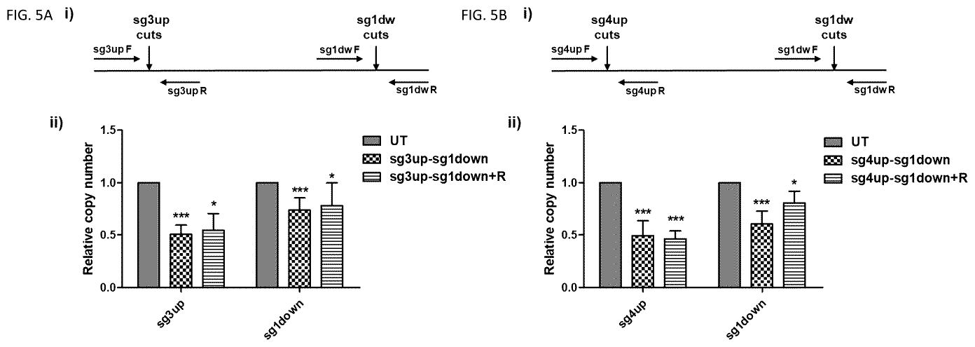

The efficiency of targeted excision of intronic TNR repeat was measured by quantitative real time PCR in vitro in cells transfected with Cas9-sgRNA (FIG. 5). Two PCR amplifications, one with a pair of primers flanking the cut site of the upstream sgRNA and another with a pair for the downstream sgRNA were performed to assess the efficiency of the dual cut across the deletion junction as shown in FIG. 5. Cells treated with dual sgRNAs in which deletion has occurred would not amplify any PCR product with either primer pair. The copy number of undeleted TNR loci was compared to that of unrelated loci elsewhere in the genome not targeted by the two sgRNA—B-actin and EGFR genes (Epidermal Growth Factor Receptor). In untreated cells the copy number of each of the three genes should be identical whereas the copy number of intact TCF4 should fall relative to B-actin and EGFR in treated cells. Relative copy number of sg3 up was calculated to be 0.5 times (50%), and that of sg1dw was 0.74 times (74%) in the sg3 up-sg1 down dual guide treated cells, compared to that of the untreated cells (FIG. 5A). Similarly, the relative copy number of sg4 up was calculated to be 0.49 times (49%), and that of sg1dw was 0.6 times (60%) in the sg4 up-sg1 down dual guide treated cells, compared to that of the untreated cells (FIG. 5A). This suggests that the TCF4 TNR was deleted in up to half of the chromosomes in transfected cells, and this is likely an underestimate as transfection efficiency is only 80% and no editing will occur in untransfected cells.

### Example 4. Materials and Methods

### gRNA Design and Synthesis

The CRISPR design tool (https://benchling.com/academic) was used to identify potential Cas9 targets lying upstream and downstream of the CTG trinucleotide expansion in the second intron of TCF4 gene and design cognate single guide RNA (sgRNA) sequences. Off-target and on-target scores were calculated using the Optimised CRISPR Design Tool and Benchling CRISPR Tool available online at http://crispr.mit.edu/ and https://benchling.com/crispr respectively. The two guide sequences upstream and downstream of the TNR with the best aggregate on-target and off-target scores were chosen (Table 1A). Synthetic sgRNAs were purchased from Synthego, USA.

TABLE 1A
TCF4 sgRNA guide sequences

| | | | | | MIT CRISPR | |
|---|---|---|---|---|---|---|
| | | | | | score | |
| | | | | | | Off |
| | | | | | | target |
| | | | | | | (no. of |
| | | | Benchling | | | genes |
| | SEQ | | score | | | in |
| sgRNA | ID | guide | On | Off | On | intronic |
| name | NO | sequence | target | target | target | region) |
| sg1up | 9 | cacttcagct | 65 | 39 | 67 | 197 |
| | | agaaaccagt | | | | |
| sg2up | 10 | gtagtctcag | 67 | 38 | 63 | 215 |
| | | tgttcagaca | | | | |
| sg1down | 13 | gaaaaacact | 70 | 36 | 58 | 326 |
| | | agtttcacca | | | | |
| sg2down | 14 | ttggccatct | 66 | 42 | 74 | 138 |
| | | aatgaacctg | | | | |

TABLE 1B
TCF4 sgRNA guide sequences

| | | | Benchling score | |
|---|---|---|---|---|
| sgRNA | SEQ | guide | On | Off |
| name | ID NO | sequence | target | target |
| sg3up | 11 | taggaaaaga | 71 | 59 |
| | | tgtaactagg | | |
| sg3up | 12 | gtcgtaggat | 74 | 43 |
| | | cagcacaaag | | |

Reporter Plasmid Preparation

50 nucleotides of WT TCF4 sequence—25 bp on both sides from the cut site for each guide sequence (Table 2) was cloned in the vector plasmid of psiTEST-LUC-Target (York Bioscience Ltd, York, UK) by standard molecular biology techniques. In brief, psiTEST-LUC-Target was digested with NheI and KpnI (NEB Cat #R0131S and #R0142S). The reporter sequences for each sgRNA were annealed and cloned into the digested plasmid.

TABLE 2
Reporter sequences cloned for TCF4 sgRNAs

| sgRNA | | SEQ ID |
|---|---|---|
| name | Reporter sequence | NO |
| sg1up | Top- | 15 |
| | aaaacccaaacacttcagctagaa | |
| | accagtaggaatctaaaggacagt | |
| | aataattttt | |
| | Bottom- | 16 |
| | CTAGaaaaattattactgtccttt | |
| | agattcctactggtttctagctga | |
| | agtgtttgggttttGTAC | |
| sg2up | Top- | 17 |
| | aactaaaccacccctaaaacttgg | |
| | ccatgtctgaacactgagactact | |
| | aatactttg | |
| | Bottom- | 18 |
| | CTAGcaaagtattagtagtctcag | |
| | tgttcagacatggccaagttttag | |
| | gggtggtttagttGTAC | |
| sg1down | Top- | 19 |
| | tatacgagatggaaatggacattg | |
| | ccacgtttatggccaaggttttca | |
| | atataaaac | |
| | Bottom- | 20 |
| | CTAGgttttatattgaaaaccttg | |
| | gccataaacgtggcaatgtccatt | |
| | tccatctcgtataGTAC | |
| sg2down | Top- | 21 |
| | gtagtactgcttggccatctaatg | |
| | aacctgaggaaaaagaaagaacag | |
| | agtgataat | |
| | Bottom- | 22 |
| | CTAGattatcactctgttctttct | |
| | ttttcctcaggttcattagatggc | |
| | caagcagtactacGTAC | |

### Preparation and In Vitro Digestion of DNA Target with Purified S. pyogenes Cas9/sgRNA

A double-stranded DNA template was prepared by amplifying a region of the reporter plasmid containing the desired target sequence using flanking primers: 5′-ACCCCAACATCTTCGACGCGGGC-3′ and 3′-TGCTGTCCTGCCCCACCCCA-5′. Cas9:sgRNA complex was formed by incubating 30 nM S. pyogenes Cas9 nuclease (NEB UK) with 30 nM synthetic sgRNA (Synthego) for 10 minutes at 25° C. The Cas9:sgRNA complex was then incubated with 3 nM of DNA template at 37° C. for 1 hour. Fragment analysis was then carried out by electrophoresis on a 1% agarose, 1×TBE gel.

Lipofection of Cas9/Synthetic RNA Ribonucleoprotein (RNP) Complexes for CRISPR Genome Editing

HEK 293 cells (Life Technologies) cells were transfected with RNP complexes consisting of purified Cas9 nuclease duplexed with synthetic guide RNA using Lipofectamine CRISPRMAX transfection kit as described in the Synthego CRISPR Transfection Protocols. Briefly, 1×10⁵ cells were seeded per well in a 24 well plate. RNP complex or RNP complex with 4.5 μg of random non-homologous single strand DNA was mixed with Lipofectamine CRISPRMAX and added to each well and incubated for 2 days at 37° C. The cells were collected and genomic DNA extracted using the QiaAMP DNA Mini Kit (Qiagen, UK). PCR and agarose gel electrophoresis were performed using specific primers (Table 3B) to detect the ability of gRNAs pairs to produce deletions of the expected size in TCF4 genomic DNA.

Real Time PCR

Primers pairs (Table 3D) were designed flanking the cut site for all the guide sequences to assess copy number variants. B-actin was used as the reference gene for normalisation. Epidermal growth factor receptor gene (EGFR) was used as an internal control gene. Copy number of the untransfected HEK293 cells were compared with that of the cells transfected with the dual sgRNAs to assess the deletion frequency.

Minigene Construct for mRNA Splicing Studies

To investigate whether deletion of the TNR sequence affects splicing of TCF4 a minigene containing TCF4 exons 2 and 3 with portions of intron 2 was constructed. Each target exon was amplified from genomic DNA from HEK293 cells by PCR using primers with a restriction site for HindIII and EcoRI for exon2 and using the primers with a restriction site for EcoRI and XhoI for exon3 fragment (Table 3C). The PCR products were cloned directly into pJET1.2 (Invitrogen, UK). Each exon fragment was excised from pJET1.2 using appropriate restriction endonucleases and directionally subcloned in a three-way ligation reaction into pcDNA 3.1. HEK 293 cells were transfected with the plasmid, using Lipofectamine 2000. After 48 h the cells were harvested, and RNA prepared using Qiagen RNeasy Mini Kit (Qiagen, UK) for use in reverse-transcription PCR (RT-PCR) that utilized multiscribe reverse-transcriptase (Applied Biosystems, UK) and a reverse pcDNA3.1 primer to synthesize the first-strand cDNA. A control reaction without reverse transcriptase was also performed.

TABLE 3A
Sequence of random oligo and site
specific single stranded DNA

| Oligo | Sequence |
|---|---|
| Random oligo (R) | Tcatgtggtcggggtagcggctg |
| | aagcactgcacgccgtacgtcag |
| | ggtggtcacgagggtgggccagg |
| | gcacgggcagcttgccggtggtg |
| | cagatgaacttcagggtcagctt |
| | gccgtaggtggc |
| | (SEQ ID NO: 23) |

TABLE 3B
Primer sequences to assess deletion
and TIDE analysis

| | | SEQ |
|---|---|---|
| Primer | | ID |
| name | Primer sequence | NO |
| sg3upF | ttctccaaggattgggactg | 24 |
| sg3upR | gctgatcctacgactacg | 25 |
| sg4upF | ggctgaatccttggtaaat | 26 |
| | atgaag | |
| sg4upR | gcagcatgaaagagccccac | 27 |
| sg1-2downF | gctggagagagagggagtg | 28 |
| sg1-2downR | cactgctcacaggaggtgaa | 29 |
| sg2downR | gagccagtaaaatgtccac | 30 |

TABLE 3C
Primer sequences for minigene cloning
and RT-PCR analysis

| | | |
|---|---|---|
| | | SEQ |
| Primer | | ID |
| name | Primer sequence | NO |

Minigene cloning primers

| | | |
|---|---|---|
| TCF4 exon2 HindIII F | atatatAAGCTTcccaac | 31 |
| | ccaacaacaagtct | |
| TCF4 exon2 EcoRIR | atatatGAATTCcacagct | 32 |
| | gttgttagtttccaccg | |
| TCF4 intron2EcoRIF | atatatGAATTCgaaaga | 33 |
| | tctttgagga | |
| TCF4 intron3 XhoIR | atatatCTCGAGcagccca | 34 |
| | gaacatttaacttaacac | |

Minigene RT-PCR primers

| | | |
|---|---|---|
| pCDNA3.1R | ctagaaggcacagtcgagg | 35 |
| Sg4up_QF | tagggacggacaaagagctg | 36 |
| TCF4 RT R1 | cactgctcacaggaggtgaa | 37 |
| sg2downR | gagccagtaaaatgtccac | 38 |

TABLE 3D
Primer sequences for real time
quantitative analysis

| | | SEQ |
|---|---|---|
| Primer | | ID |
| name | Primer sequence | NO |
| Sg3up_QF | tagggacggacaaagagctg | 39 |
| Sg3up_QR | ccaaggattcagccaattaaa | 40 |
| Sg4up_QF | | |
| Sg4down_QR | | |
| Sg1down_QF | gcctccaacctgaatcttga | 41 |
| Sgldown_QR | aaaagagaaacaacattacagatcc | 42 |
| b-actin_QF | tcacccacactgtgcccatctacga | 43 |
| b-actin_QR | cagcggaaccgctcattgccaatgg | 44 |
| EGFR_QF | ccagtattgatcgggagagccgga | 45 |
| EGFR_QR | cttttcctccagagcccgactcgc | 46 |

1. A method of altering a gene product, the method comprising:
administering into a cell an engineered CRISPR/Cas9 system comprising at least one vector comprising:
(i) a nucleotide molecule encoding Cas9 nuclease;
(ii) a first sgRNA comprising a first CRISPR targeting RNA (crRNA) sequence that hybridizes to a nucleotide sequence complementary to a first target sequence in a first intron, the first target sequence being positioned 5′ of a disease-causing repeat expansion that is present in the first intron; and
(iii) a second sgRNA comprising a second crRNA sequence that hybridizes to a nucleotide sequence complementary to a second target sequence in the first intron, the second target sequence being positioned 3′ of the disease-causing repeat expansion,
wherein the at least one vector does not have a nucleotide molecule encoding Cas9 nuclease and a crRNA sequence that naturally occur together.

2. The method according to claim 1, wherein the first and second target sequences are positioned 5′ and 3′, respectively, of the intronic CTG18.1 trinucleotide repeat expansion of TCF4.

3. The method according to claim 1, wherein at least one of the first and second crRNA sequences comprises a nucleotide sequence having at least 85% sequence identify to a sequence selected from the group consisting of guide sequences shown in Table 1A or Table 1B.

4. The method according to claim 1, wherein at least one of the first and second crRNA sequences comprises a nucleotide sequence selected from the group consisting of guide sequences shown in Table 1A or Table 1B.

5. The method according to claim 1, wherein
the first crRNA sequence comprises the first target sequence;
the second crRNA sequence comprises the second target sequence;
the first crRNA sequence is from 17 to 24 nucleotide long; and/or
the second crRNA sequence is from 17 to 24 nucleotide long.

6. The method according to claim 1, wherein the first and/or second PAMs and the Cas9 nuclease are from Streptococcus or Staphylococcus.

7. The method according to claim 1, wherein the first and second PAMs are both from Streptococcus or Staphylococcus.

8. The method according to claim 1, wherein each of the first and second PAMs independently consists of NGG or NNGRRT, wherein N is any of A, T, G, and C, and R is A or G.

9. The method according to claim 1, wherein the administering comprises injecting the engineered CRISPR/Cas9 system into the cell.

10. The method according to claim 1, wherein the administering comprises introducing the engineered CRISPR/Cas9 system into a cell containing and expressing a DNA molecule having the target sequence.

11. The method according to claim 1, including:
administering the engineered CRISPR/Cas9 system into a subject.

12. The method according to claim 11, wherein the subject is a human.

13. The method according to claim 12, wherein the disease is Fuchs' endothelial corneal dystrophy (FECD).

14. The method according to claim 11, further comprising:
prior to administering to a subject the engineered CRISPR/Cas9 system:
obtaining sequence information of the subject; and
selecting the first crRNA sequence and/or the second crRNA sequence based on the sequence information of the subject.

15. The method according to claim 14, wherein the sequence information of the subject includes whole-genome sequence information of the subject.

16. The method according to claim 1, wherein the method prevents, ameliorates or treats a disease associated with a repeat expansion in a subject in need thereof.

17. A method of treating a disease associated with a repeat expansion in a subject in need thereof, comprising:
(a) obtaining a plurality of stem cells comprising a nucleic acid mutation in a corneal dystrophy target nucleic acid from the subject;
(b) manipulating the nucleic acid mutation in one or more stem cells of the plurality of stem cells to correct the nucleic acid mutation, thereby forming one or more manipulated stem cells;
(c) isolating the one or more manipulated stem cells; and
(d) transplanting the one or more manipulated stem cells into the subject, wherein manipulating the nucleic acid mutation in the one or more stem cells of the plurality of stem cells includes performing the method of claim 1.

18. A single guide RNA (sgRNA) comprising a nucleotide sequence having at least 85% sequence identify to a sequence selected from the group consisting of guide sequences shown in Table 1A or Table 1B.

19. The sgRNA according to claim 18, comprising a sequence selected from the group consisting of guide sequences shown in Table 1A or Table 1B.

20. An engineered Clustered Regularly Interspaced Short Palindromic Repeats (CRISPR)/CRISPR associate protein 9 (Cas9) system comprising (i) at least one vector comprising a nucleotide molecule encoding Cas9 nuclease and the sgRNA of claim 18.
